(19) United States
(12) Patent Application Publication
(10) Pub. No.: US 2020/0035342 A1
(43) Pub. Date: Jan. 30, 2020

(54) Neural Network Encoders and Decoders for Claim Adjustment

(71) Applicant: drchrono Inc., Sunnyvale, CA (US)

(72) Inventors: Daniel Kivatinos, Mountain View, CA (US); Michael Nusimow, Mountain View, CA (US); Martin Borgt, Sunnyvale, CA (US); Soham Waychal, Sunnyvale, CA (US)

(21) Appl. No.: 16/457,778

(22) Filed: Jun. 28, 2019

Related U.S. Application Data
(60) Provisional application No. 62/703,877, filed on Jul. 27, 2018.

(51) Int. Cl. G16H 15/00; G06N 3/04; G06N 3/08

(52) U.S. Cl. CPC G16H 15/00; G06N 3/08; G06N 3/0445; G06N 3/0454

(57) ABSTRACT

A machine learning system may be trained to assist physicians with claims by automatically adjusting the claims to make them more likely to be accepted by a payer or by outputting a predicted probability that the claim will be accepted. The machine learning system may use one or more encoders that encode codes, clinical notes, and claims into separate vector spaces, where the vector spaces relate similar entities. The encoded codes, clinical notes, and claims may be decoded by a decoder to predict codes comprising an adjusted claim. Alternatively, the decoder may output a predicted probability that the claim will be accepted for payment. The encoders and the decoder may be machine learning models that are trained using ground-truth training examples.

### CROSS-REFERENCE TO RELATED APPLICATIONS

This application claims the benefit of U.S. Provisional Patent Application No. 62/703,877, filed Jul. 27, 2018, which is hereby incorporated by reference in its entirety.

### FIELD OF THE INVENTION

The present invention relates to using machine learning encoders and decoders to automatically suggest adjustments to a physician claim or predict the probability that a claim will be accepted by a payer.

### BACKGROUND

When physicians bill for a treatment, such as a procedure or office visit, they record the treatments using standardized codes. These standardized codes are submitted to the payer, such as an insurance company, Medicaid, or Medicare. For example, standardized codes may be used for procedures like examining the patient's knee or setting a broken arm. Codes are sometimes numeric or alphanumeric. Some code systems include CPT, ICD-9, ICD-10, SNOMED, LOINC, RxNorm, HCPCS, and others. Some code systems are more specialized for diagnosis, while others are more specialized for procedures and payment. Some code systems have as many as tens of thousands of codes.

A claim may be submitted by a physician for a single office visit by patient and may include multiple codes, one for each treatment or procedure performed during the visit. In some cases, procedures that to a lay person seem to be a single procedure are billed as sets of multiple codes.

The process of choosing the right codes to include in a claim for a procedure or treatment is often opaque and difficult. Payers may deny or reject claims that do not include the right combinations of codes. However, it is difficult for physicians to choose the “correct” codes to include in a claim because payers provide little guidance and there are often a large number of potential codes that could be used. Moreover, physicians do not know how likely it is for claims that they submit to be accepted for payment.

### SUMMARY OF THE INVENTION

Some embodiments relate to a machine learning system for adjusting a billing claim to be more likely to be accepted for payment by a payer. The machine learning system may be used prior to submission of the billing claim to make it more likely that the billing claim will be accepted in the first instance. In other embodiments, the machine learning system may be used to adjust a billing claim after it has been submitted to a payer and rejected. The machine learning system may then assist the physician in adjusting the claim so that it can be resubmitted to the payer.

In other embodiments, a machine learning system is used to output a predicted probability that a billing claim will be accepted for payment by a payer.

In one embodiment, a billing code encoder is provided that encodes billing codes into a first vector representation. In one embodiment, a clinical note encoder is provided that encodes clinical notes into a second vector representation. In one embodiment, a billing claim encoder is provided that encodes a billing claim into a third vector representation. Each vector representation relates similar entities in a vector space by locating them closer together and causes dissimilar entities to be farther apart in the vector space.

In one embodiment, a billing claim and an associated clinical note are provided. The individual billing codes in the billing claim are encoded using the billing code encoder. The text and diagnosis codes in the clinical note may also be encoded. The billing claim may be encoded by the billing claim encoder, and the clinical note may be encoded by the clinical note encoder. The encoded billing claim and the encoded clinical note may be input to a decoder. In some embodiments, the decoder outputs one or more predicted billing codes comprising an adjusted billing claim. In some embodiments, the decoder outputs a predicted probability that the billing claim will be accepted for payment by the payer.

In one embodiment, the encoders and the decoder are machine learning models that are trained using ground-truth training examples.

### BRIEF DESCRIPTION OF THE DRAWINGS

FIG. 1 illustrates an exemplary machine learning system that may be used in some embodiments.

FIG. 2 illustrates an exemplary method of training the machine learning system.

FIG. 3 illustrates an exemplary method for using the machine learning system to output an adjusted billing claim or a predicted probability that a billing claim is accepted for payment.

FIG. 4A illustrates an exemplary method for training a billing code encoder.

FIG. 4B illustrates an exemplary method for encoding a billing code.

FIG. 5A illustrates an exemplary method for training a clinical note encoder.

FIG. 5B illustrates an exemplary method for encoding a clinical note.

FIG. 6A illustrates an exemplary method for training a billing claim encoder.

FIG. 6B illustrates an exemplary method for encoding a billing claim.

FIG. 7A illustrates an exemplary method of training a decoder.

FIG. 7B illustrates an exemplary method of outputting predicted billing codes or a probability of acceptance of a billing claim from the decoder.

FIG. 8 illustrates an exemplary skip-gram neural network.

FIG. 9 illustrates an exemplary long short-term memory (LSTM) neural network.

FIG. 10 illustrates an exemplary triplet loss neural network.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments relate to using one or more machine learning models to adjust a billing claim of a physician. The adjustment may be performed before submission of the billing claim to a payer to increase chances of acceptance, or after a billing claim was submitted and denied in order to adjust the billing claim to be resubmitted and accepted. The recommended adjustment may be made based on the billing codes in the billing claim and a clinical note associated with the billing claim. The machine learning models may be trained based on billing claims and clinical notes of other physicians, such as physicians who practice in a similar field or see similar patients.

In other embodiments, the machine learning models are used to output a predicted probability of acceptance of the billing claim by the payer, rather than an adjusted claim.

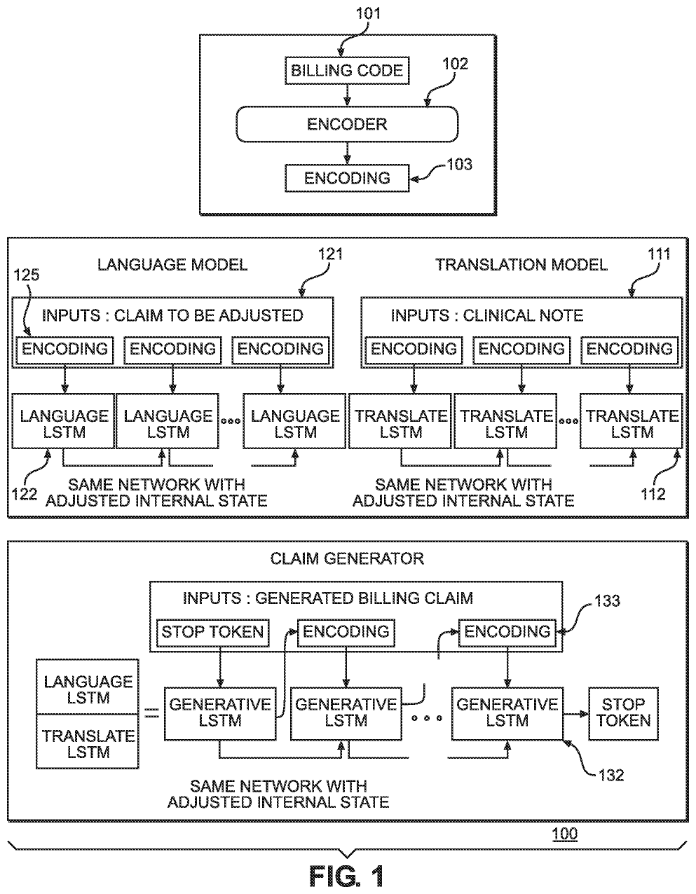

FIG. 1 illustrates an exemplary machine learning system 100 for adjusting a billing claim to increase its probability for acceptance. Illustrated variations of machine learning system 100 may also be used for predicting the probability that a billing claim will be accepted. System 100 may be embodied on a single computer system or on multiple computer systems. Encodings may be used to encode, or translate, raw text into representations that encode more information than the raw text, so that machine learning models have more information to train on. Raw text alone often fails to capture relationships with other similar concepts. Therefore, encoded representations are used herein to map concepts into a vector space. The encoded representations relate similar concepts so that similar concepts are located closer together in the vector space and unrelated concepts are located farther from each other in the vector space. This is accomplished by training a machine learning model to minimize the vector distance between similar concepts and maximize the vector distance between dissimilar concepts. Vector distance may be measured by cosine similarity, dot product, or other distance metrics.

In some embodiments, billing codes 101 are mapped into a vector representation, or encoding. In raw format, billing codes 101 may simply be numeric or alphanumeric, and therefore the raw format does not capture relationships between billing codes 101, such as the fact that billing codes 28630 and 28515 may be for related procedures that are often performed together.

A billing code encoder 102 may accept one or more billing codes 101 as input, in their raw format, and output a vector representation for each of the billing codes 101, where the vector representation relates similar billing codes. The vector representations comprise the encoded billing codes 103. The billing code encoder 102 may be implemented with a neural network. An encoder comprising a neural network may be referred to herein as a neural network encoder. In some embodiments, the encoding used for billing codes is word embedding. Some forms of word embedding include skip-gram, continuous bag of words, and Word2Vec. Billing codes may have attached information such as price, allowed price by the payer, and so forth, in addition to the treatment or procedure code itself.

A clinical note 111 includes text comprising doctor notes from a patient visit and codes representing patient events, for example diagnosis codes that represent diagnoses in a standardized code system. For use in machine learning, it is desirable to encode the clinical note 111 to relate similar clinical notes in vector space.

The codes, which may initially be provided in a raw text format, may be encoded prior to encoding the clinical note 111. Codes may be encoded using the same method as billing code encoder 102, except using a separately trained model because the codes in the clinical note 111 will generally not be billing codes, but other kinds of codes, such as diagnosis codes. A diagnosis code encoder (not illustrated), may be implemented in the same manner as billing code encoder 102, and trained on diagnosis codes instead of billing codes. For example, a skip-gram neural network may be used for the encoding.

Keywords may be extracted from the text of the clinical note 111. The keywords may be identified using statistical techniques like term frequency-inverse document frequency or by unsupervised techniques like clustering, Latent Dirichlet Analysis, or Latent Semantic Analysis. Keywords of the text may then be encoded using word embedding, such as using a skip-gram neural network model, or through a triplet loss neural network model.

Clinical note encoder 112 may accept as input a clinical note 111 comprising one or more encoded codes and encoded keywords of text and output an encoding of the clinical note. The encoded clinical note may comprise a fixed length vector representation of the variable length clinical note 111, and the vector representation may relate similar clinical notes in vector space. In some embodiments, the clinical note encoder 112 is a neural network. In some embodiments, the clinical note encoder 112 is a recurrent neural network (RNN). In some embodiments, the clinical note encoder 112 is a long short-term memory (LSTM) neural network, which is one type of RNN.

A billing claim 121 may also be used in system 100. A billing claim comprises a set of one or more billing codes submitted by the physician in one claim, which may correspond to a single visit by a patient to the physician's office. For example, if a patient received a general examination, an examination of their ankle, and setting of a broken ankle during a visit, then billing codes for each of these procedures may be included in a single billing claim. A billing claim may be associated with a clinical note that includes the physician's written notes from the patient visit corresponding to the billing claim.

The billing claim 121 may initially be provided as set of billing codes in raw text format. However, the billing codes may be input to the billing code encoder 102 to create encoded billing codes 125 of the billing claim 121. The billing claim encoder 122 then receives as input the billing claim 121 and outputs encoded billing claim. The encoded billing claim may comprise a vector representation that relates similar billing claims in vector space. The encoded billing claim may be a fixed length vector representation. In some embodiments, the billing claim encoder 122 is a neural network. In some embodiments, the billing claim encoder 122 is an RNN. In some embodiments, the billing claim encoder 122 is an LSTM neural network.

The encoded clinical note and encoded billing claim may be combined, such as by concatenating the two vectors into a single vector. The vector may be input to a decoder 132, which generates output 133. In some embodiments, output 133 is an adjusted billing claim, comprising one or more billing codes. In other embodiments, output 133 is a predicted probability that the payer will accept the billing claim for payment. The concatenation, or other combination, of vectors permits the decoder 132 to use information from both the clinical note and the associated billing claim to make its prediction. In some embodiments, the decoder 132 is a neural network. In some embodiments, the decoder 132 is an RNN. In some embodiments, the decoder 132 is an LSTM neural network.

Decoder 132 may output an adjusted billing claim in the form of one or more encoded billing codes, the encoded billing codes in a vector representation in the same format and vector space used by the encoded billing codes 103. The decoder 132 may then map the encoded version of the billing codes to a raw format to provide the actual billing codes themselves. This may be accomplished by using a similarity metric, such as cosine similarity, to find the most similar encoded billing code to each predicted vector according to the similarity metric, and the decoder 132 predicting the most similar encoded billing code to the predicted vector as the output.

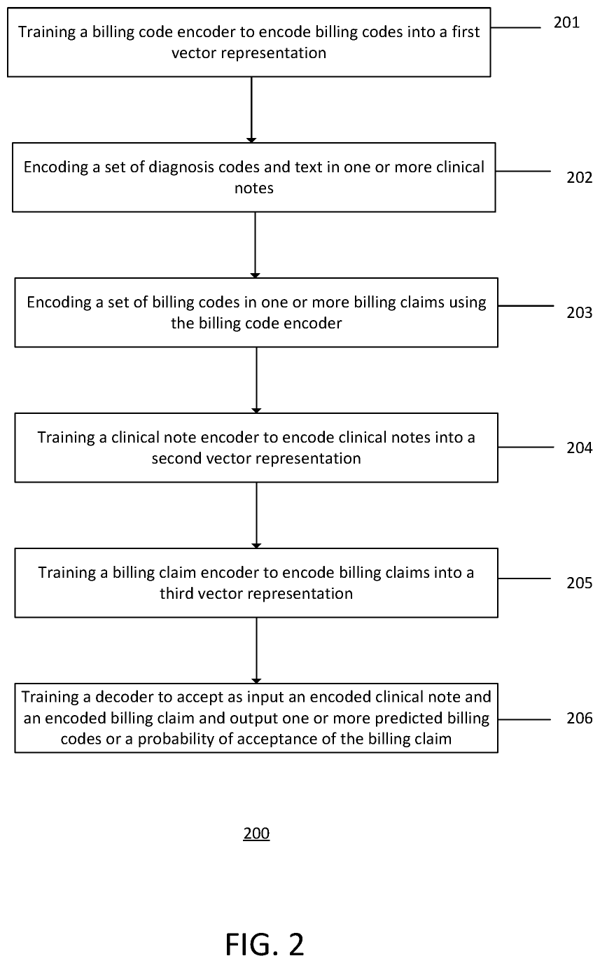

FIG. 2 illustrates an exemplary method 200 for training machine learning system 100 that may be used in an embodiment. The training data for the machine learning system 100 may include one or more ground-truth examples of billing claims that were successfully accepted by a payer. Moreover, for training a machine learning system 100 to predict the probability that a billing claim will be accepted by the payer, one or more ground-truth examples of billing claims that were rejected by the payer may also be used for training.

In step 201, a billing code encoder may be trained to encode billing codes 101 into a first vector representation.

In step 202, a set of diagnosis codes in raw format in a clinical note may be encoded using the diagnosis code encoder. The text of the clinical note may also be encoded by extracting keywords and encoding them with word embeddings.

In step 203, a set of billing codes in raw format in a billing claim may be encoded using the billing code encoder 102 to create billing claim 121 represented with encoded billing codes 125. The clinical note and the billing claim are associated, and the clinical note may describe the visit for which the billing claim was submitted.

In step 204, a clinical note encoder is trained to encode clinical notes into a second vector representation.

In step 205, a billing claim encoder is trained to encode billing claims into a third vector representation.

The first vector representation, second vector representation, and third vector representation may each be distinct from each other. The first vector representation is in a vector space of billing codes. The second vector representation is in a vector space of clinical notes. The third vector representation is in a vector space of billing claims.

In step 206, decoder 132 may be trained to accept as input an encoded clinical note 111 and an encoded billing claim 121 and produce output 133. In some embodiments, output 133 is one or more predicted billing codes comprising an adjusted billing claim. The decoder 132 may be trained to produce a stop token to end the sequence of billing codes comprising the adjusted billing claim. In other embodiments, output 133 is a predicted probability that the payer will pay the billing claim. Backpropagation may be used to train decoder 132 to reduce, or minimize, the error between the actual output of the decoder 132 and the desired output.

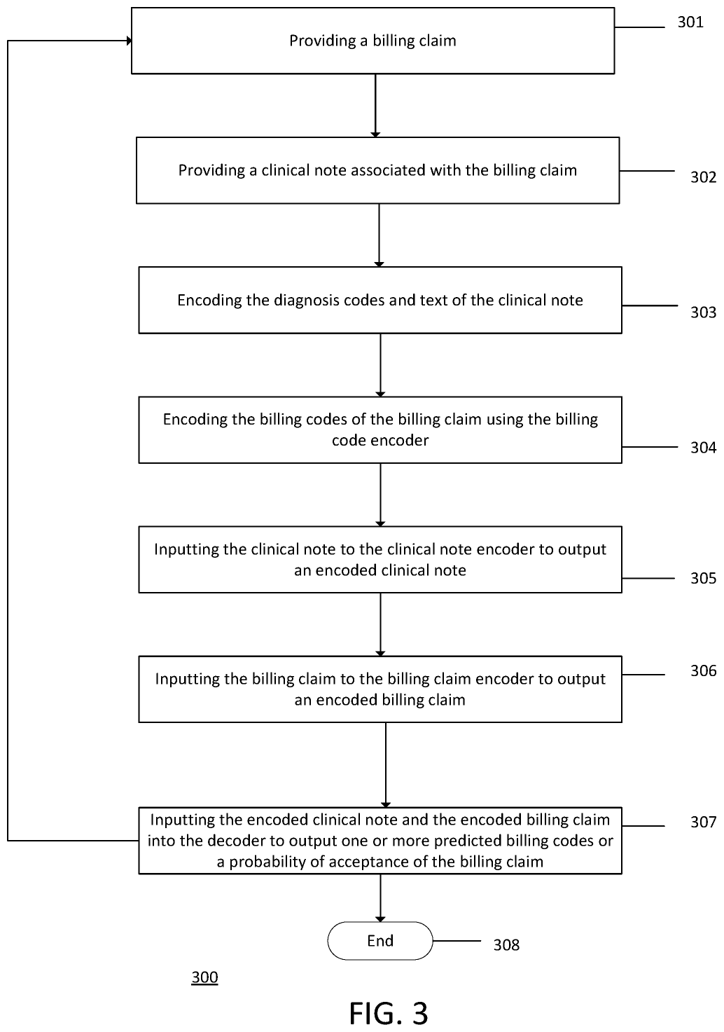

FIG. 3 illustrates an exemplary method that may be used in an embodiment. Method 300 is a method of using machine learning system 100 to produce an adjusted billing claim, or to output a predicted probability of acceptance of the billing claim, based on a billing claim and a clinical note. In step 301, a billing claim may be provided, where the billing codes of the billing claim may initially be in raw format.

In step 302, a clinical note associated with the billing claim may be provided, which may comprise text and diagnosis codes in raw text format.

In step 303, the diagnosis codes of the clinical note may be encoded using a diagnosis code encoder. The text of the clinical note may be encoded by identifying keywords and encoding them with an encoder, such as using word embeddings.

In step 304, the billing codes of the billing claim may be encoded using the billing code encoder 102. This outputs the billing claim in the form of a set of encoded billing codes.

In step 305, the clinical note, comprising encoded diagnosis codes and encoded keywords, may be input to the clinical note encoder 112 to output an encoded clinical note.

In step 306, the billing claim, comprising a set of encoded billing codes, may be input to the billing claim encoder 122 to output an encoded billing claim.

In step 307, the encoded clinical note and the encoded billing claim may be combined, such as by concatenation. The combined vector may be input to the decoder 132.

In one embodiment, the decoder 132 outputs one or more predicted billing codes that comprise an adjusted billing claim, or a stop token.

If the decoder 132 outputs the stop token, then the method ends at step 308. However, if the decoder 132 outputs a predicted billing code, then the method may continue by adding the predicted billing code to an adjusted billing claim and then repeating method 300 from step 301. The method 300 may repeat iteratively, adding a newly predicted billing codes to the adjusted billing claim until a stop token is output by the decoder 132 or a maximum length is reached.

In some embodiments, the adjusted billing claim is created by adding the predicted billing codes to the input billing claim provided in step 301. In this embodiment, the input billing claim is the starting point for the adjusted billing claim. To avoid simply adding billing codes and allow the possibility of modifying the input billing claim by modifying or deleting billing codes, the input billing claim may be re-initialized to be empty or to have only a few of its billing codes prior to starting method 300. The billing claim may then be effectively rebuilt using method 300. For each billing code known to be in the original input billing claim, the probability that the decoder 132 outputs that billing code may be increased, prior to initiating method 300, in order to increase the likelihood that the adjusted billing code will converge in a manner to be similar to the real input billing code.

In some embodiments, beam search is used to follow multiple potential paths of adding predicted billing codes. The use of beam search to follow multiple paths aids in avoiding the method getting stuck at a local maximum without reaching a potentially higher maximum elsewhere. Beam search is an algorithm with a configurable branching factor n, which defines the number of paths that are followed.

When beam search is used, in step 307 the decoder 132 outputs a vector representation of a billing code or a stop token. A set of n billing codes are chosen to be added in separate branches of the beam search. In some embodiments, the n closest billing codes to the output vector representation are chosen. However, other methods of selection are also possible, such as adding a small random number to the output vector representation and then choosing the closest billing codes to obtain more diversity of outcomes. Another iteration of method 300 is performed for each of the n most promising branches.

At each iteration, the n most promising additions of predicted billing codes are added to the adjusted billing claim, each in a separate branch of the search, and the beam search continues to iterate. The beam search ends when all branches have terminated with a stop token or have reached a maximum length. When the beam search has ceased, the algorithm outputs the set of billing codes—the adjusted billing claim—having the highest predicted probability.

In some embodiments, rather than outputting billing codes, the decoder 132 is trained to output a probability that the billing claim will be accepted by the payer for payment. In this case, the input billing claim is the full billing claim to be evaluated and is not re-initialized to be empty or have only a subset of billing codes as described in the prior embodiment. The billing code is input in full to the machine learning system 100 and, through method 300, the decoder outputs probability of acceptance of the billing claim.

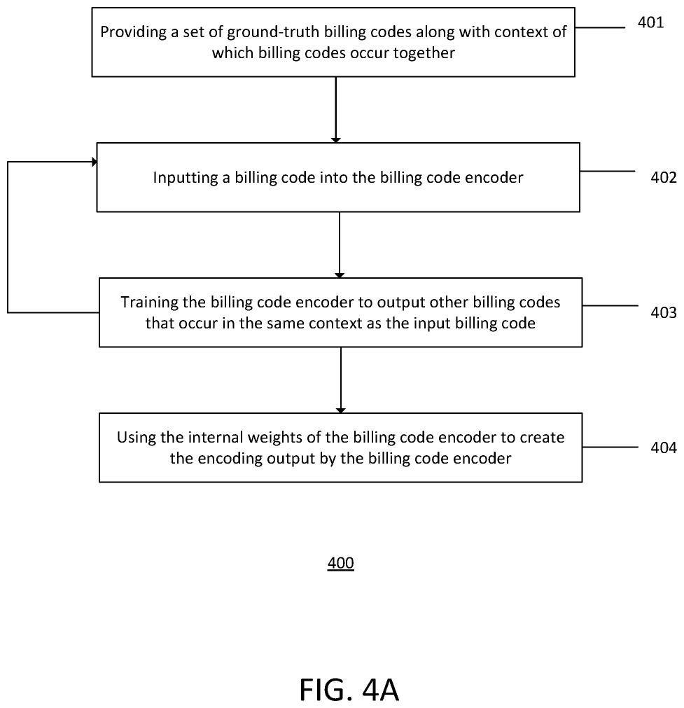

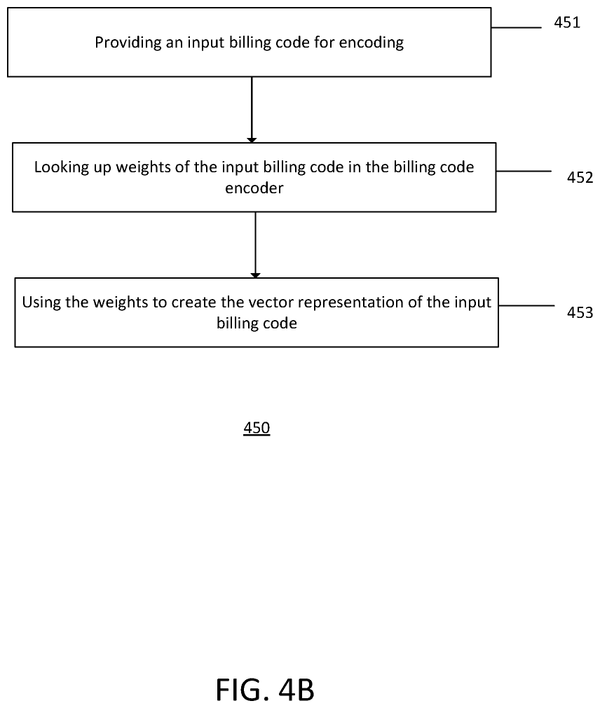

FIG. 4A illustrates an exemplary method 400 for training the billing code encoder 102, which may be used in some embodiments.

In step 401, a set of ground-truth billing codes is provided along with context of which billing codes occur together. For example, the ground-truth billing codes may be provided as a set of millions of billing claims that were submitted by physicians, or that were accepted for payment by a payer. Through method 400, the billing code encoder 102 is trained to output vector representations so that billing codes that commonly occur together are encoded to be closer together in the vector space than billing codes that do not commonly occur together.

In step 402, a billing code is input into the billing code encoder 102. In some embodiments, the billing code encoder 102 uses the skip-gram model. In the skip-gram model, the billing code is input as a one-hot encoding. A one-hot encoding is a vector with all the entries of the vector equal to ‘0’ except for a single ‘1.’ Each entry in the vector represents a single item, and the location of the ‘1’ indicates the item that is represented by the vector. Therefore, a one-hot encoding of billing codes means that the vector has length equal to the number of billing codes and each entry represents a single billing code. In the skip-gram model, the billing code encoder 102 may be a single layer neural network.

In step 403, the billing code encoder 102 is trained to output other billing codes that occur in the same claim as the input billing code. This may be performed by inputting the other billing codes that occur in the claims as a correct output for the input to billing code encoder 102 and performing backpropagation in the neural network comprising the billing code encoder 102. The output billing codes may also be represented by one-hot encodings. Steps 402 and 403 may be repeated for multiple billing codes.

In step 404, in a skip-gram model, the internal weights of the billing code encoder 102 may be used to generate the output encoding of the billing code encoder 102. In some embodiments, the set of internal weights for the input billing code may be used as the vector representation. In some embodiments, the set of internal weights of the input billing code may be pairwise added to the internal weights for the input billing code in the decoding step of the skip-gram neural network to generate the vector representation.

FIG. 4B illustrates an exemplary method 450 for encoding a billing code that may be used in some embodiments.

In step 451, an input billing code in raw format may be provided for encoding. The input billing code may be transformed into a one-hot encoding.

In step 452, the internal weights in the billing code encoder 102 for the input billing code may be retrieved.

In step 453, the weights may be used by the billing code encoder 102 to generate the vector representation of the input billing code. In some embodiments, the set of internal weights for the input billing code may be used as the vector representation. In some embodiments, the set of internal weights of the input billing code may be pairwise added to the internal weights for the input billing code in the decoding step of the skip-gram neural network to generate the vector representation.

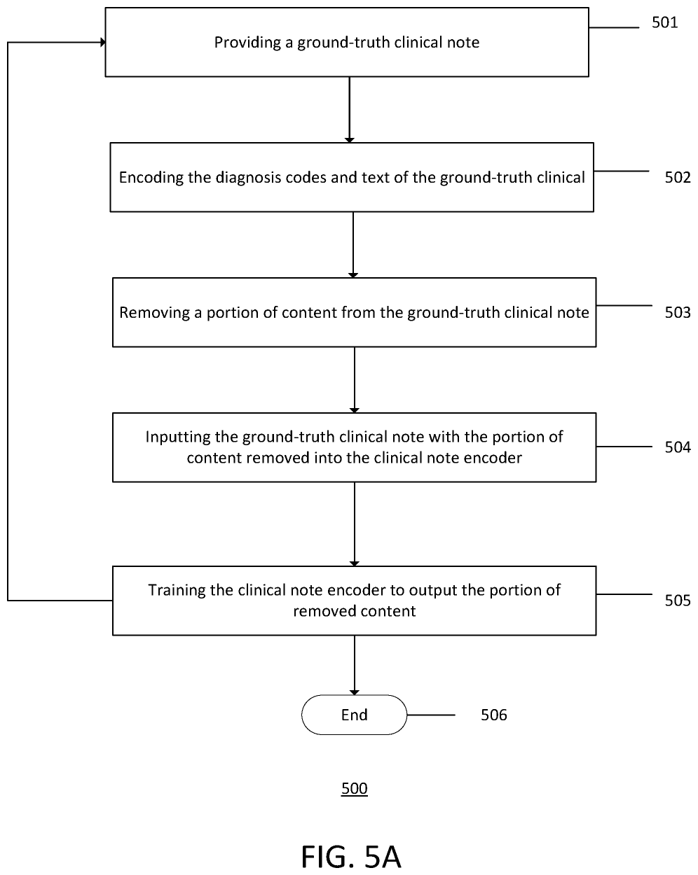

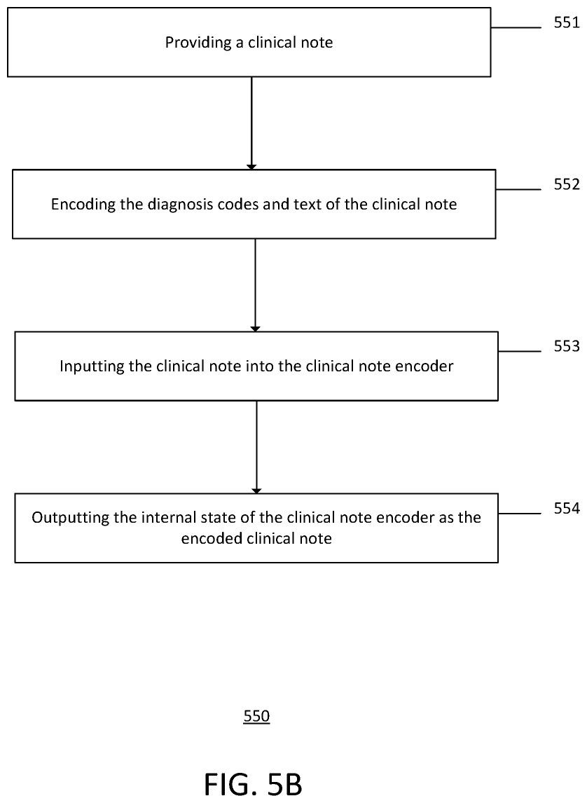

FIG. 5A illustrates an exemplary method 500 for training the clinical note encoder 112, which may be used in some embodiments.

In step 501, a ground-truth clinical note may be provided. The ground-truth clinical note is a known clinical note that is used for training.

In step 502, the diagnosis codes and text of the ground-truth clinical note may be encoded. The diagnosis codes may be encoded using a diagnosis code encoder, and the text may be encoded by extracting keywords and using word embeddings. Optionally, the diagnosis codes may be randomly reordered and likewise the text keywords may be randomly reordered during training to reduce or eliminate the effect of order on training.

In step 503, a portion of content may be removed from the ground-truth clinical note. For instance, the portion of content removed may be one or more diagnosis codes or one or more keywords representing the text. In this way, the ground-truth clinical note is now missing a portion of content that would correctly be included in the clinical note.

In step 504, the ground-truth clinical note with the portion of content removed is input into the clinical note encoder 112.

In step 505, the clinical note encoder 112 is trained to output the portion of content that was removed in vector form. For example, it may be trained to output the removed diagnosis codes or removed keywords, in their vector representations. This is performed by inputting the encoded versions of the removed content as correct outputs of the clinical note encoder 112 when the clinical note is input in step 504. Backpropagation is used to reduce the error between the actual output of the clinical note encoder 112 and the encoded versions of the removed portion of content, which is the target output.

The method 500 may repeat at step 501 to train on additional clinical notes. The method 500 ends at step 506.

FIG. 5B illustrates an exemplary method 550 of encoding a clinical note that may be used in some embodiments.

In step 551, a clinical note may be provided with the diagnosis codes and text in raw format.

In step 552, the diagnosis codes and text of the clinical note may be encoded. The diagnosis codes may be encoded using a diagnosis code encoder and the clinical note may be encoded by extracting keywords and encoding them using word embeddings.

In step 553, the clinical note, comprising encoded diagnosis codes and encoded keywords, may be input into the clinical note encoder 112. In some embodiments, the clinical note encoder 112 is an LSTM, which stores an internal state that is preserved through iterations of the LSTM. In the LSTM, the internal state is stored as a vector and transmitted as input into the next iteration of the LSTM, when a new token is input.

In step 554, the clinical note encoder 112 may output the internal state of the clinical note encoder as the encoded clinical note. In some embodiments, the internal state of the clinical note encoder that is output is the internal state of an LSTM neural network or is based on the internal state of the LSTM neural network.

In some embodiments, a triplet loss neural network may be used to further improve encodings produced by the clinical note encoder 112 by locating similar clinical notes more closely together in vector space and unrelated clinical notes farther from each other in vector space. A triplet loss neural network may be a neural network with one or more layers. The triplet loss neural network may be initially trained to output for a given clinical note the encoding provided by the LSTM neural network, skip-gram, or other encoding model. This provides a triplet loss neural network that takes as input a clinical note and outputs an encoding. The triplet loss method may then be used to train the triplet loss neural network based on use of an Anchor, a Positive, and a Negative. The triplet loss method uses gradient descent to change the weights of the triplet loss neural network to reduce the encoding distance between the Anchor and the Positive and increase the encoding distance between the Anchor and the Negative.

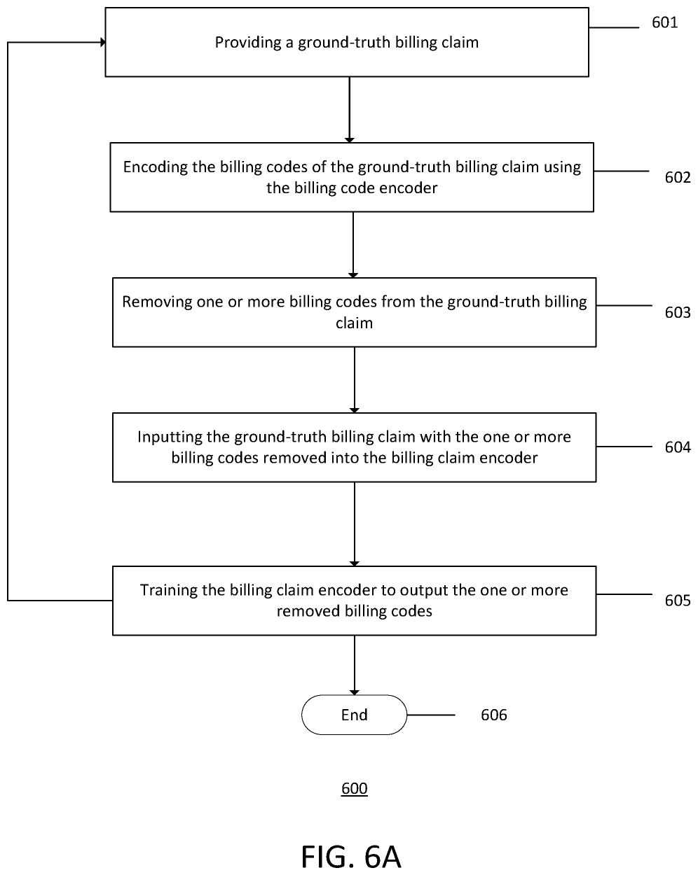

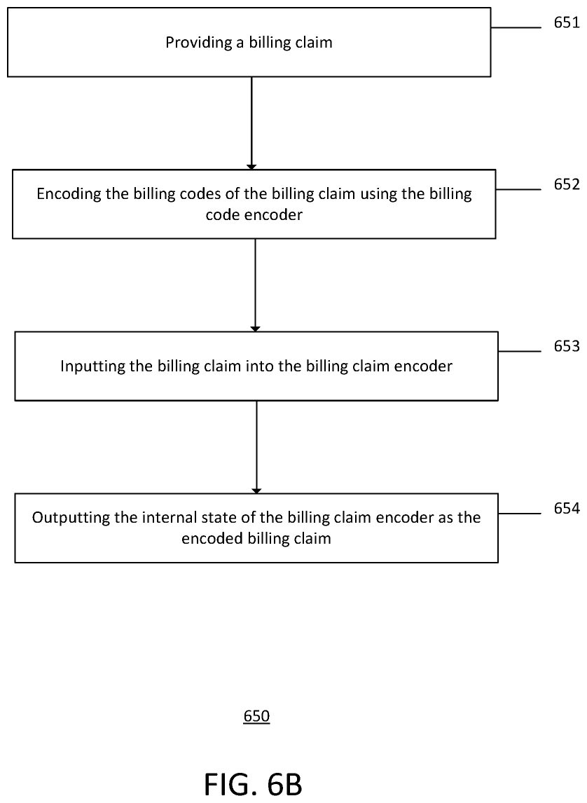

FIG. 6A illustrates an exemplary method 600 for training the billing claim encoder 122, which may be used in some embodiments.

In step 601, a ground-truth billing claim may be provided. The ground-truth billing claim is a known billing claim that is used for training.

In step 602, the billing codes of the ground-truth billing claim may be encoded using billing code encoder 102 to create encoded billing codes. Optionally, the billing codes of the billing claim may be randomly reordered during training to reduce or eliminate the effect of billing code order on the training.

In step 603, one or more billing codes may be removed from the ground-truth billing claim. In this way, the ground-truth billing claim is now missing one or more billing codes that would correctly be included in the ground-truth billing claim.

In step 604, the ground-truth billing claim with the one or more billing codes removed is input into the billing claim encoder 122.

In step 605, the billing claim encoder 122 is trained to output the one or more billing codes that were removed in encoded vector form. This is performed by inputting the encoded versions of the removed billing codes as correct outputs of the billing claim encoder 122 when the encoded ground-truth billing claim is input in step 604. Backpropagation is used to reduce error between the actual output of the billing claim encoder 122 and the encoded versions of the removed billing codes, which are the target output.

FIG. 6B illustrates an exemplary method 650 of encoding a billing claim that may be used in some embodiments.

In step 651, a billing claim may be provided with the billing codes in raw format.

In step 652, the billing codes of the billing claim may be encoded using the billing code encoder 102 so that each billing code of the billing claim is encoded.

In step 653, the billing claim, comprising encoded billing codes, may be input into the billing claim encoder 122. In some embodiments, the billing claim encoder 122 is an LSTM, which stores an internal state that is preserved through iterations of the LSTM. In the LSTM, the internal state is stored as a vector and transmitted as input into the next iteration of the LSTM, when a new token (or in this case billing code) is input.

In step 654, the billing claim encoder 122 may output the internal state of the billing claim encoder as the encoded billing claim. In some embodiments, the internal state of the billing claim encoder 122 that is output is the internal state of an LSTM neural network or is based on the internal state of the LSTM neural network.

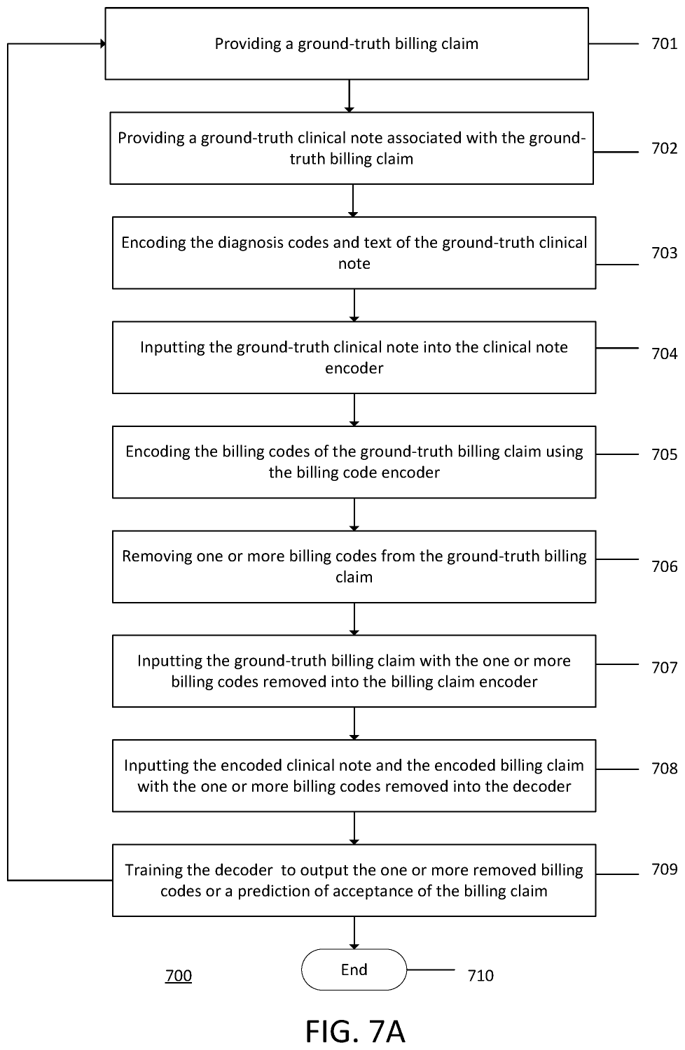

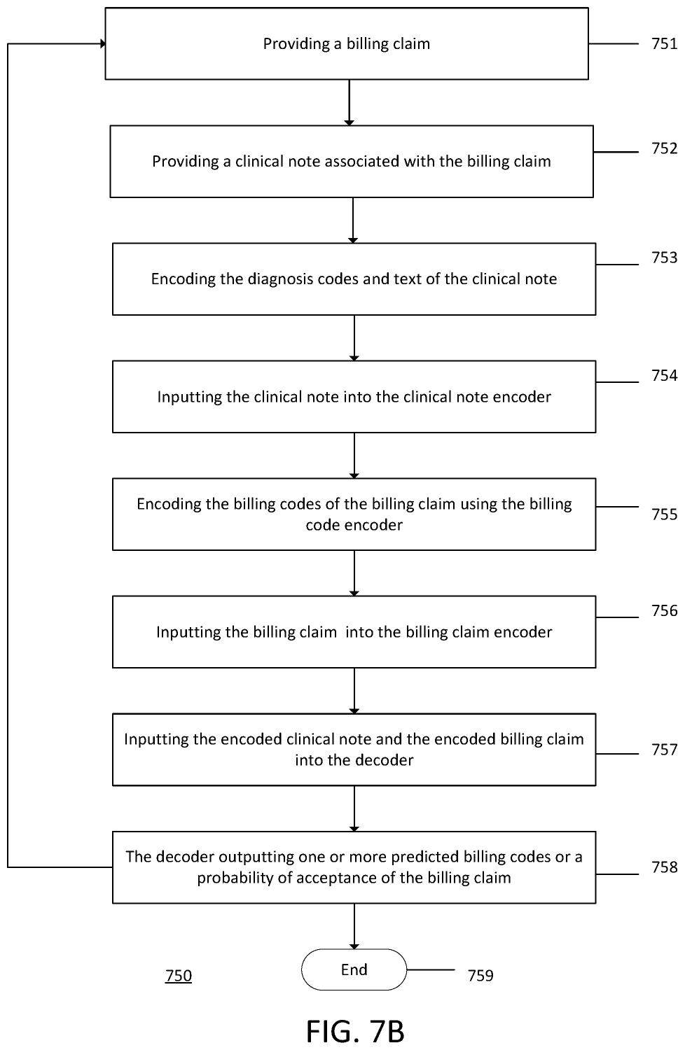

FIG. 7A illustrates an exemplary method 700 of training the decoder 132 to correctly predict billing codes, or probability of acceptance of the billing claim, which may be used in some embodiments.

In step 701, a ground-truth billing claim may be provided.

In step 702, a ground-truth clinical note associated with the ground-truth billing claim may be provided.

In step 703, the diagnosis codes and text of the clinical note may be encoded. The diagnosis codes may be encoded using a diagnosis code encoder. The text of the clinical note may be encoded by extracting keywords and using word embeddings on the keywords.

In step 704, the ground-truth clinical note may be input into the clinical note encoder 112 to output an encoded clinical note.

In step 705, the billing codes of the billing claim may be encoded using the billing code encoder 102. This produces a billing claim comprising encoded billing codes.

In step 706, one or more billing codes are removed from the ground-truth billing claim.

In step 707, the ground-truth billing claim with the one or more billing codes removed are input into the billing claim encoder 122 to output an encoded billing claim with one or more billing codes removed.

In step 708, the encoded clinical note and the encoded billing claim may be combined, such as by concatenation. The resulting vector may be input into the decoder 132.

In step 709, the decoder 132 is trained to output the one or more removed billing codes. This is performed by inputting the encoded versions of the removed billing codes as correct outputs of the decoder 132 when the encoded clinical note and encoded billing claim with one or more billing codes removed are input in step 708. Backpropagation is used to reduce error between the actual output of the decoder 132 and the encoded versions of the removed billing codes, which are the target output.

Alternatively, the decoder 132 may be trained to instead output a probability that the billing claim will be accepted by the payer. In this embodiment, no billing codes are removed from the ground-truth billing claim. Thus, step 706 is not performed and steps 707-709 use the full ground-truth billing claim with no billing codes removed. In step 709, the decoder 132 is trained to output the correct result value of accepted or rejected based on the ground-truth result of whether the input billing claim was accepted or rejected. The output may be represented by a single output layer of the decoder with two nodes, one representing accepted and the other representing rejected. An output of ‘1’ at either node represents a 100% probability that the claim was accepted or rejected. Fractional values are used to represent probabilities that the claim was accepted or rejected, and the values must sum to ‘1’. Backpropagation is used to reduce error between the actual output of the decoder 132 and the correct output of accepted or rejected.

FIG. 7B illustrates an exemplary method 750 of using decoder 132, after decoder 132 has been trained, to output predicted billing codes comprising an adjusted billing claim, or a probability that the billing claim is accepted for payment.

In step 751, an input billing claim is provided. In some embodiments, the billing claim is input for adjustments. In other embodiments, the billing claim is input to predict the probability that it will be accepted for payment.

In step 752, a clinical note associated with the input billing claim is provided.

In step 753, the diagnosis codes and text of the clinical note are encoded. The diagnosis codes are encoded using the diagnosis code encoder and the text is encoded by extracting keywords and encoding them, such as by using word embeddings.

In step 754, the clinical note is input to the clinical note encoder 112 to output an encoded clinical note.

In step 755, the billing codes of the billing claim are encoded using the billing code encoder 102.

In step 756, the billing claim, comprising encoded billing codes, is input into the billing claim encoder 122 to output an encoded billing claim.

In step 757, the encoded clinical note and the encoded billing claim are combined, such as by concatenation. The resulting vector is input into the decoder 132.

In step 758, the decoder 132 outputs one or more billing codes, or a predicted probability of acceptance of the billing claim.

If the decoder 132 has been trained to output billing codes, then the decoder 132 outputs vector representations of one or more billing codes and maps the vector representations to the original format of the billing codes by finding the most similar billing code in the vector space. The predicted billing code may be added to an adjusted billing claim and the process repeated. If the decoder 132 outputs a stop token or reaches a maximum length of billing codes in an adjusted billing claim, then the process ends at step 759. Beam search may be used to follow multiple possible paths and avoid being stuck in a local maximum.

If the decoder 132 has been trained to output probabilities of acceptance of the billing claim, then the decoder 132 outputs a number between 0 and 1 representing the probability that the billing claim will be accepted for payment. Conversely, the decoder 132 may also output a probability that the billing claim will be denied or rejected.

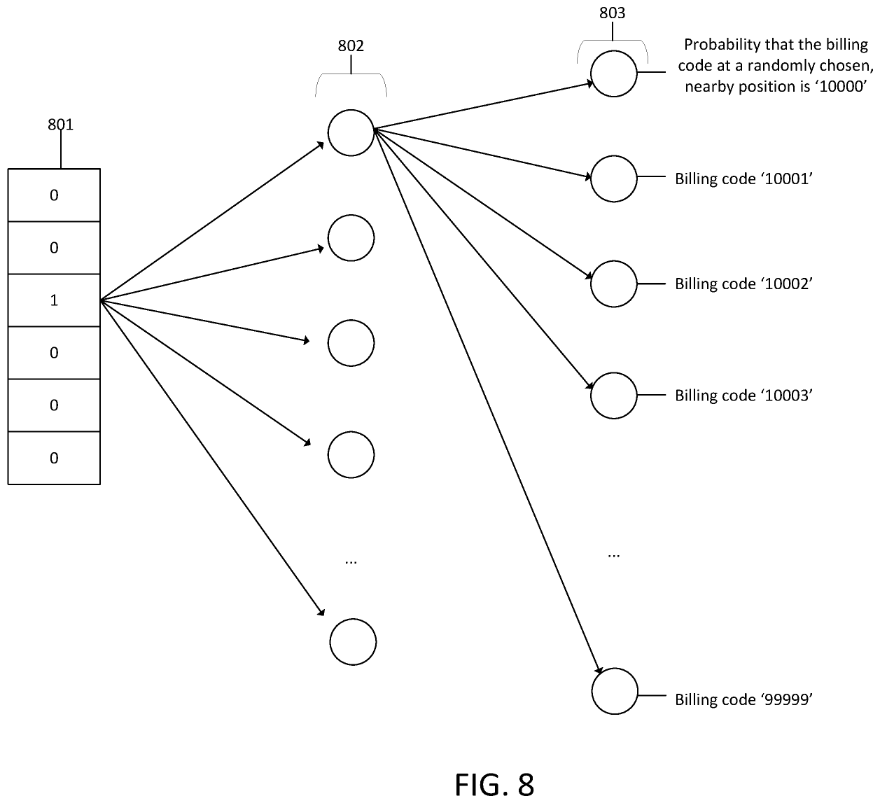

FIG. 8 illustrates a skip-gram model, which may be used in some embodiments to implement billing code encoder 102, diagnoses code encoder, or word embeddings for text.

In the context of the billing code encoder 102, the skip-gram model is a one layer neural network trained to accept one-hot encoding of billing codes as inputs and output a probability distribution of the likely billing codes to appear in the same claim. A one-hot encoding of a billing code 801 is accepted as the input to a single hidden layer of neurons 802. The single hidden layer is connected to an output layer of neurons 803. Each layer is fully-connected. The hidden layer of neurons 802 may use a linear activation function for their output, and the output layer of neurons 803 may use the Softmax function as the activation function. After training the skip-gram model on ground-truth examples of billing codes and other billing codes appearing in the same billing claim, the weights in the hidden layer 802 for each one-hot encoding may be used as the vector representation for the corresponding billing code of the one-hot encoding.

The illustrated skip-gram model may be used for diagnosis codes by using one-hot encodings of diagnosis codes as inputs and outputs rather than billing codes. Likewise, the model may also be used for word embeddings of text. The input and output in that case would be one-hot encodings of text words, and the single layer neural network would be trained to output words that are likely to appear in the same context with the input word, meaning with a window of n words from the input word. The value n may be fixed or configurable.

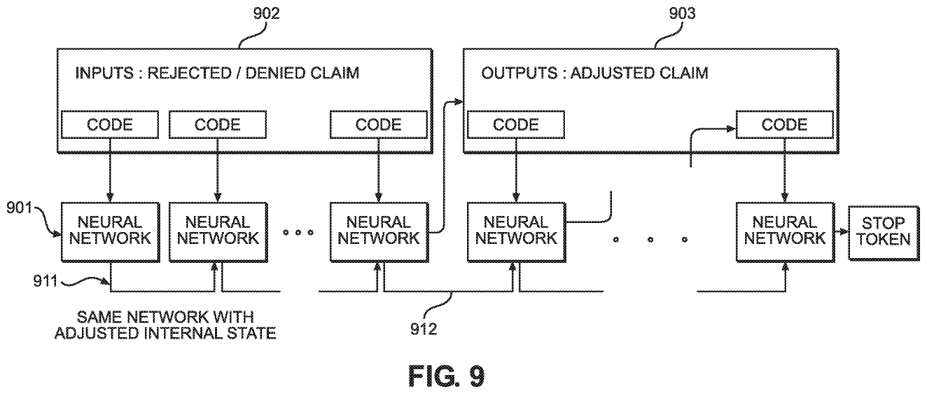

FIG. 9 illustrates an LSTM neural network, which may be used to implement the clinical note encoder 112, billing claim encoder 122, and decoder 132. The LSTM neural network 901 comprises a plurality of neural network nodes. The neural network nodes may have an activation function that defines the output of the neural network nodes based on the input to the neural network node. Some of the neural network nodes may have a sigmoid activation function and other of the neural network nodes may have a tan h (hyperbolic tangent function) activation function. The LSTM neural network 901 may also have one or more gates to control the flow of information. Some of the gates may perform pointwise addition, and other of the gates may perform pointwise multiplication.

When used to implement the billing claim encoder 122, the LSTM neural network accepts input 902 comprising a sequence of billing codes. As described above, the billing codes may be encoded using the billing code encoder 102. The billing codes 902 are input sequentially to the LSTM neural network 901. At each step, the LSTM neural network 901 takes as input the next billing code from billing codes 902, and an internal state passed from the prior iteration, such as state 911 passed from the first iteration to the second iteration in the diagram. The internal state may be represented as a vector. At each iteration, the LSTM neural network 901 outputs internal state and also an output representing a billing code, in an encoded vector representation, or a stop token.

The output billing codes are ignored until the last code of the input 902 has been input to the LSTM neural network 901. At that point, the billing code output by the LSTM neural network 901 becomes part of the output 903, which may be predicted billing codes to be included in the adjusted billing claim. The output billing codes 903 may be added to the adjusted billing claim and the process continued to predict additional output billing codes 903 that may also be added to the adjusted billing claim. The process may end when a stop token or a maximum length is reached.

During the encoding process, the internal state vector 912 output by the LSTM neural network 901 after the last input billing code 902 has been input may be the vector representation used for the encoded billing claim, when using the billing claim encoder 122.

When used to implement the clinical note encoder 112, the input to the LSTM may be a sequence of tokens comprising encoded keywords representing the text of the clinical note and encoded diagnosis codes. The output may be tokens representing encoded keywords and encoded diagnosis codes that are related to the clinical note. The internal state 912 at the last iteration of the input may be used as an encoding of the clinical note.

In some embodiments, the LSTM neural network 901 may output a probability of acceptance of the billing claim 902, rather than a set of predicted billing codes. The output layer of the LSTM neural network 901 is changed from encoding a vector representation of a billing code to instead having two nodes, one for acceptance and one for rejection. After the last billing code of the billing claim 902 is input, the output of the neural network 901 is the predicted probability that the billing claim as a whole is accepted or rejected.

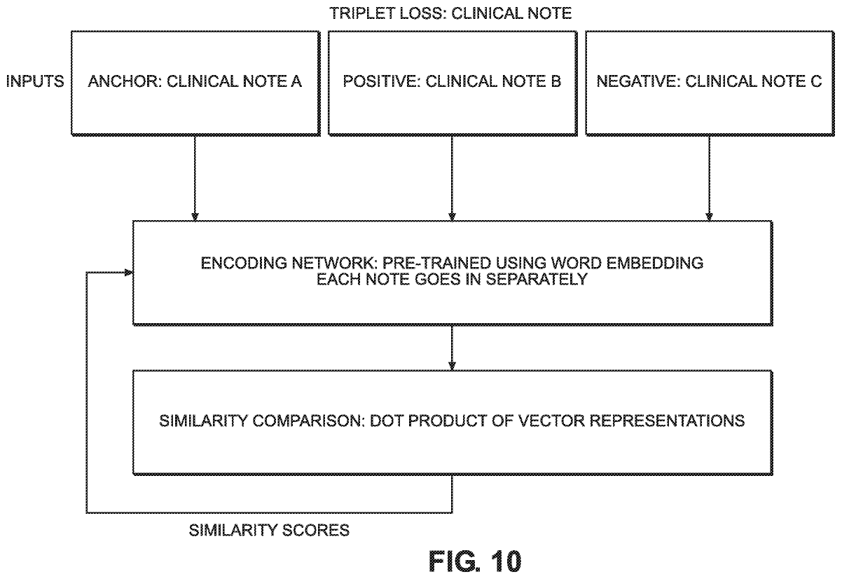

FIG. 10 illustrates a triplet loss neural network, which may be used to encode clinical notes and other entities. Triplet loss may be used to train a neural network to output similar encodings when two inputs are similar in a desired dimension and reduce the similarity of encodings when two inputs are similar in undesired dimensions.

A triplet loss neural network may have one or more layers of neural network nodes that each have inputs, outputs, and an activation function. The triplet loss neural network may be initially trained to output correct encodings of entities, such as clinical notes, where the initial encodings are obtained based on other methods of encoding such as skip-gram or an LSTM neural network. For example, the neural network may be initially trained to output, for an entity, the encoding learned from a skip-gram neural network model or LSTM neural network model. Triplet loss may then be used to refine the encoding to make similar entities closer and make dissimilar entities farther away in the encoding. Alternatively, the triplet loss neural network is not initialized to output an encoding and the triplet loss neural network may initially encode entities to essentially random encodings. The triplet loss method may then be used to train the triplet loss neural network to learn and output correct encodings.

In the triplet loss method, three training examples are input into the triplet loss neural network, the Anchor (A), the Positive (P), and the Negative (N). The Anchor is the reference input. The Positive is an input that should have a similar encoding to the Anchor, but which appears different in the input data. The Negative is an input that should have a dissimilar encoding to the Anchor, but which appears similar in the input data. One example is the Anchor being a clinical note written by Doctor 1 about Procedure A, the Positive being a clinical note written by Doctor 2 about Procedure A, and the Negative being a clinical note written by Doctor 1 about Procedure B. The triplet loss neural network is trained using gradient descent using a loss function of form L(A, P, N)=max(∥f(A)−f(P)∥²−∥f(A)−f(N)∥²+α, 0) where α is a margin. By minimizing the loss function, the triplet loss neural network is trained to encode the Anchor and Negative to be farther apart when measured under a distance metric and encode the Anchor and Positive to be closer to together when measured under a distance metric.

While the triplet loss method has been illustrated with encodings of clinical notes, the triplet loss method may also be used for training neural network encoders for other entities like keywords, billing codes, diagnosis codes, and billing claims.

The terminology used herein is for the purpose of describing particular aspects only and is not intended to be limiting of the disclosure. As used herein, the singular forms “a,” “an,” and “the” are intended to comprise the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

While the invention has been particularly shown and described with reference to specific embodiments thereof, it should be understood that changes in the form and details of the disclosed embodiments may be made without departing from the scope of the invention. Although various advantages, aspects, and objects of the present invention have been discussed herein with reference to various embodiments, it will be understood that the scope of the invention should not be limited by reference to such advantages, aspects, and objects. Rather, the scope of the invention should be determined with reference to patent claims.

What is claimed:

1. A computer-implemented method for using a plurality of neural network encoders to recommend adjustments to a claim, the method comprising:
training a first neural network encoder to encode codes into a first vector representation, the first vector representation relating codes that are similar;
training a second neural network encoder to encode clinical notes into a second vector representation, the second vector representation relating clinical notes that are similar;
training a third neural network encoder to encode claims into a third vector representation, the third vector representation relating claims that are similar, wherein the claims comprise one or more codes;
training a neural network decoder to accept as input an encoded clinical note and an encoded claim to output one or more predicted codes;
generating an adjusted claim by:
providing a claim;
providing a clinical note;
inputting the clinical note to the second neural network encoder to output an encoded clinical note;
inputting the claim to the third neural network encoder to output an encoded claim;
inputting the encoded clinical note and the encoded claim into the neural network decoder to output the adjusted claim, the adjusted claim comprising one or more codes.

2. The computer-implemented method of claim 1, wherein the first neural network encoder creates word embeddings of codes by using a skip-gram model.

3. The computer-implemented method of claim 1, wherein the second neural network encoder is a long short-term memory neural network (LSTM), and
the second vector representation is created based on the internal state of the second neural network encoder.

4. The computer-implemented method of claim 1, wherein the second neural network encoder is trained by:
removing a portion of content from a ground-truth clinical note;
inputting the ground-truth clinical note with the portion of content removed into the second neural network encoder; and
training the second neural network encoder to output the removed portion of content.

5. The computer-implemented method of claim 1, wherein the third neural network encoder is a long short-term memory (LSTM) neural network, and
the third vector representation is created based on the internal state of the third neural network encoder.

6. The computer-implemented method of claim 1, wherein the third neural network encoder is trained by:
removing one or more codes from a ground-truth claim;
inputting the ground-truth claim with the one or more codes removed into the third neural network encoder; and
training the third neural network encoder to output the one or more removed codes.

7. The computer-implemented method of claim 1, wherein the neural network decoder is trained by:
providing a ground-truth claim;
providing a ground-truth clinical note associated with the ground-truth claim;
inputting the ground-truth clinical note into the second neural network encoder to output an encoded ground-truth clinical note;
removing one or more codes from the ground-truth claim;
inputting the ground-truth claim with the one or more codes removed into the third neural network encoder to output an encoded ground-truth claim with the one or more codes removed;
inputting the encoded ground-truth clinical note and the encoded ground-truth claim with the one or more codes removed in the neural network decoder, and
training the neural network decoder to output the one or more removed codes.

8. A computer-implemented method for recommending adjustments to a claim, the method comprising:
training a first encoder to encode codes into a first vector representation, the first vector representation relating codes that are similar;
training a second encoder to encode clinical notes into a second vector representation, the second vector representation relating clinical notes that are similar;
training a third encoder to encode claims into a third vector representation, the third vector representation relating claims that are similar, wherein the claims comprise one or more codes;
training a decoder to accept as input an encoded clinical note and an encoded claim to output one or more predicted codes;
generating an adjusted claim by:
providing a claim;
providing a clinical note;
inputting the clinical note to the second encoder to output an encoded clinical note;
inputting the claim to the third encoder to output an encoded claim;
inputting the encoded clinical note and the encoded claim into the decoder to output the adjusted claim, the adjusted claim comprising one or more codes.

9. The computer-implemented method of claim 8, wherein the first encoder creates word embeddings of codes by using a skip-gram model.

10. The computer-implemented method of claim 8, wherein the second encoder is a long short-term memory neural network (LSTM), and
the second vector representation is created based on the internal state of the second encoder.

11. The computer-implemented method of claim 8, wherein the second encoder is trained by:
removing a portion of content from a ground-truth clinical note;
inputting the ground-truth clinical note with the portion of content removed into the second encoder; and
training the second encoder to output the removed portion of content.

12. The computer-implemented method of claim 8, wherein the third encoder is a long short-term memory (LSTM) neural network, and
the third vector representation is created based on the internal state of the third encoder.

13. The computer-implemented method of claim 8, wherein the third encoder is trained by:
removing one or more codes from a ground-truth claim;
inputting the ground-truth claim with the one or more codes removed into the third encoder; and
training the third encoder to output the one or more removed codes.

14. The computer-implemented method of claim 8, wherein the decoder is trained by:
providing a ground-truth claim;
providing a ground-truth clinical note associated with the claim;
inputting the ground-truth clinical note into the second encoder to output an encoded ground-truth clinical note;
removing one or more codes from the ground-truth claim;
inputting the ground-truth claim with the one or more codes removed into the third encoder to output an encoded ground-truth claim with the one or more codes removed;
inputting the encoded ground-truth clinical note and the encoded ground-truth claim with the one or more codes removed into the decoder, and
training the decoder to output the one or more removed codes.

15. A computer-implemented method for recommending adjustments to a claim, the method comprising:
training a first encoder to encode codes into a first vector representation;
training a second encoder to encode clinical notes into a second vector representation;
training a third encoder to encode claims into a third vector representation, wherein the claims comprise one or more codes;
training a decoder to accept as input an encoded clinical note and an encoded claim to output one or more predicted codes;
generating an adjusted claim by:
providing a claim;
providing a clinical note;
inputting the clinical note to the second encoder to output an encoded clinical note;
inputting the claim to the third encoder to output an encoded claim;
inputting the encoded clinical note and the encoded claim into the decoder to output the adjusted claim, the adjusted claim comprising one or more codes.

16. The computer-implemented method of claim 15, wherein the first encoder creates word embeddings of codes by using a skip-gram model.

17. The computer-implemented method of claim 15, wherein the second encoder is a long short-term memory neural network (LSTM), and
the second vector representation is created based on the internal state of the second encoder.

18. The computer-implemented method of claim 15, wherein the second encoder is trained by:
removing a portion of content from a ground-truth clinical note;
inputting the ground-truth clinical note with the portion of content removed into the second encoder; and
training the second encoder to output the removed portion of content.

19. The computer-implemented method of claim 15, wherein the third encoder is a long short-term memory (LSTM) neural network, and
the third vector representation is created based on the internal state of the third encoder.

20. The computer-implemented method of claim 15, wherein the third encoder is trained by:
removing one or more codes from a ground-truth claim;
inputting the ground-truth claim with the one or more codes removed into the third encoder; and
training the third encoder to output the one or more removed codes.
